(19) United States
(12) Patent Application Publication
(10) Pub. No.: US 2005/0085822 A1
(43) Pub. Date: Apr. 21, 2005

(54) Surgical navigation system component fault interfaces and related processes

(76) Inventors: Robert C. Thornberry, Tallahassee, FL (US); Jody Stallings, Palm Harbor, FL (US)

Correspondence Address: CHIEF PATENT COUNSEL;SMITH & NEPHEW, INC., 1450 BROOKS ROAD, MEMPHIS, TN, 38116, US

(21) Appl. No.: 10/689,103

(22) Filed: Oct. 20, 2003

(51) Int. Cl. A61F 5/00; A61B 19/00

(52) U.S. Cl. 606/86; 606/130

(57) ABSTRACT

Devices and processes for use in computer aided or computer navigated surgery include one or more fault interfaces interposed between an indicium and an item to be used during surgery such as a body part, tool, implant, trial or other structure or component. After the indicia have been registered into the system and surgery begun, it is sometimes the case that indicia can be inadvertently moved or dislodged in position and/or orientation relative to the body part. Fault interfaces according to various embodiments are designed to fail first, so that the indicia can be repositioned relative to the item without the need to reregister the indicia into the system relative to the item. The fault interfaces preferably include structure that allows the indicium to be repositioned relative to the item so that it does not need to be reregistered into the system.

### FIELD OF THE INVENTION

The present invention relates to frame attachments for use in surgical navigation, and methods for their use. More specifically, the invention relates to frame attachments comprising fiducials or other reference structures which are designed to be accurately reinstalled into correct position if inadvertently or otherwise moved or altered with respect to their original registration in a surgical navigation system.

### BACKGROUND

A major concern during surgical procedures as well as other medical operations is carrying out the procedures with as much precision as possible. For example, in orthopedic procedures, less than optimum alignment of implanted prosthetic components may cause undesired wear and revision, which may eventually lead to the failure of the implanted prosthesis. Other general surgical procedures also require precision in their execution.

With orthopedic procedures, for example, previous practices have not allowed for precise alignment of prosthetic components. For example, in a total knee arthroplasty, previous instrument design for resection of bone limited the alignment of the femoral and tibial resections to average value for varus/valgus, flexion/extension and external/internal rotation. Additionally, surgeons often use visual landmarks or “rules of thumb” for alignment which can be misleading due to anatomical variability. Intramedullary referencing instruments also violate the femoral and tibial canal. This intrusion increases the risk of fat embolism and unnecessary blood loss in the patient.

Devices and processes according to various embodiments of the present invention are applicable not only for knee repair, reconstruction or replacement surgery, but also repair, reconstruction or replacement surgery in connection with any other joint of the body as well as any other surgical or other operation where it is useful to track position and orientation of body parts, non-body components and/or virtual references such as rotational axes, and to display and output data regarding positioning and orientation of them relative to each other for use in navigation and performance of the operation.

Several manufacturers currently produce image-guided surgical navigation systems that are used to assist in performing surgical procedures with greater precision. The TREON™ and iON™ systems with FLUORONAV™ software manufactured by Medtronic Surgical Navigation Technologies, Inc. are examples of such systems. The BrainLAB VECTORVISION™ system is another example of such a surgical navigation system. Systems and methods for accomplishing image-guided surgery are also disclosed in U.S. Ser. No. 10/364,859, filed Feb. 11, 2003 and entitled “Image Guided Fracture Reduction,” which claims priority to U.S. Ser. No. 60/355,886, filed Feb. 11, 2002 and entitled “Image Guided Fracture Reduction”; U.S. Ser. No. 60/271,818, filed Feb. 27, 2001 and entitled “Image Guided System for Arthroplasty”; U.S. Ser. No. 10/229,372, filed Aug. 27, 2002 and entitled “Image Computer Assisted Knee Arthroplasty”; U.S. Ser. No. 10/084,278 filed Feb. 27, 2002 and entitled “Total Knee Arthroplasty Systems and Processes,” which claims priority to provisional application entitled “Surgical Navigation Systems and Processes,” Ser. No. 60/355,899, filed Feb. 11, 2002; U.S. Ser. No. 10/084,278 filed Feb. 27, 2002 and entitled “Surgical Navigation Systems and Processes for Unicompartmental Knee Arthroplasty,” which claims priority to provisional application entitled “Surgical Navigation Systems and Processes,” Ser. No. 60/355,899, filed Feb. 11, 2002; U.S. Ser. No. 10/084291 entitled Surgical Navigation Systems and Processes for High Tibial Osteotomy,” which claims priority to provisional application entitled “Surgical Navigation Systems and Processes,” Ser. No. 60/355,899, filed Feb. 11, 2002; provisional application entitled “Image-guided Navigated Precisions Reamers,” Ser. No. 60/474,178, filed May 29, 2003; and nonprovisional application entitled “Surgical Positioners,” T. Russell, P. Culley, T. Ruffice, K. Raburn and L. Grisoni, inventors, filed Oct. 3, 2003, the entire contents of each of which are incorporated herein by reference as are all documents incorporated by reference therein.

These systems and processes use position and/or orientation tracking sensors such as infrared sensors acting stereoscopically or other sensors acting in conjunction with reference structures or reference transmitters to track positions of body parts, surgery-related items such as implements, instrumentation, trial prosthetics, prosthetic components, and virtual constructs or references such as rotational axes which have been calculated and stored based on designation of bone landmarks. Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated reference structures such as fiducials, reference transmitters, or based on stored position and/or orientation information. The processing functionality correlates this position and orientation information for each object with stored information, such as a computerized fluoroscopic imaged file, a wire frame data file for rendering a representation of an instrument component, trial prosthesis or actual prosthesis, or a computer generated file relating to a rotational axis or other virtual construct or reference. The processing functionality then displays position and orientation of these objects on a screen or monitor, or otherwise. Thus, systems or processes, by sensing the position of reference structures or transmitters, can display or otherwise output useful data relating to predicted or actual position and orientation of body parts, surgically related items, implants, and virtual constructs for use in navigation, assessment, and otherwise performing surgery or other operations.

Some of these reference structures or reference transmitters may emit or reflect infrared light that is then detected by an infrared camera. The references may be sensed actively or passively by infrared, visual, sound, magnetic, electromagnetic, x-ray or any other desired technique. An active reference emits energy, and a passive reference merely reflects energy. Reference structures may have at least three, but usually four, markers or fiducials that are traced by an infrared sensor to determine the position and orientation of the reference and thus the position and orientation of the associated instrument, implant component or other object to which the reference is attached.

In addition to reference structures with fixed fiducials, modular fiducials, which may be positioned independent of each other, may be used to reference points in the coordinate system. Modular fiducials may include reflective elements which may be tracked by two, sometimes more sensors whose output may be processed in concert by associated processing functionality to geometrically calculate the position and orientation of the item to which the modular fiducial is attached. Like fixed fiducial reference structures, modular fiducials and the sensors need not be confined to the infrared spectrum—any electromagnetic, electrostatic, light, sound, radio frequently or other desired technique may be used. Similarly, modular fiducials may “actively” transmit reference information to a tracking system, as opposed to “passively” reflecting infrared or other forms of energy.

Some image-guided surgical navigation systems allow reference structures to be detected at the same time the fluoroscopy imaging is occurring. This allows the position and orientation of the reference structure to be coordinated with the fluoroscope imaging. Then, after processing position and orientation data, the reference structures may be used to track the position and orientation of anatomical features that were recorded fluoroscopically. Computer-generated images of instruments, components, or other structures that are fitted with reference structures may be superimposed on the fluoroscopic images. The instruments, trial, implant or other structure or geometry can be displayed as 3-D models, outline models, or bone-implant interface surfaces.

Some image-guided surgical navigation systems monitor the location and orientation of the reference structures and consequently the portion of the anatomy or instruments secured to the reference structure by either actively or passively detecting the position of fiducials associated with the reference structure. Because the fiducials may be arranged in particular patterns, the system can determine the exact orientation and location of the reference structure associated with the fiducials. In other words, depending upon the particular location of the individual fiducials, the system will “see” the reference structure in a particular way and will be able to calculate the location and orientation of the reference structure based upon that data. Consequently, the system can determine the exact orientation and location of the portion of the anatomy or instrument associated with the reference structure.

The exact spatial relationship of the individual fiducials with respect to each other and the associated anatomy or instrument forms the basis of how a fiducial-based system calculates the position and orientation of the associated items. Similarly, the exact spatial relationship of a reference transmitter with respect to its associated anatomy or instrument forms the basis of how a transmitter-based system calculates the position and orientation of the associated anatomy or instruments. Consequently, once the spatial relationship of the fiducials or reference transmitter with respect to the associated item to be tracked has been registered in the system, subsequent changes in the position and/or orientation of the fiducials or reference transmitter may cause the system to erroneously calculate the position and orientation of the anatomy or instruments associated with the fiducials or reference transmitter. Even minor changes in orientation and/or position of the references may lead to dramatic differences in how the system detects the orientation and/or location of the associated anatomy or instruments. Such changes may require the system to be recalibrated, requiring additional fluoroscopy or other imaging to be obtained, increasing the time and the expense of the procedure. Failure to recalibrate the system may lead to imprecision in the execution of the desired surgical procedure.

In a busy operating room, there is a possibility that reference structures, or one or more fiducials on a reference structure, will be inadvertently deformed or displaced in position or orientation, such as by a surgeon or nurse's arm or elbow, after calibration. When this happens, the reference structures and/or fiducials will provide inaccurate information about the location, position, and orientation of the body parts, non-body components and other reference points previously placed in the coordinate system and the accuracy and safety of the surgical procedure may be jeopardized. Even where a surgeon or other surgery attendant tries to place the reference structure back in its original position, it is virtually impossible to relocate the original location, position and orientation with precision. And as discussed above, even the slightest change can have dramatic results.

As a result, when a reference structure or fiducial loses its original position in the reference system, the entire coordinate system must be recalibrated or reregistered. To continue with the image guided surgery, the surgeon must reregister each instrument that will be used in the procedure and each reference structure and fiducial that is on the patient or otherwise in the coordinate system. This process lengthens the time necessary to complete the surgical procedure and can result in unnecessary complications resulting from the additional length of time the patient is in surgery.

Adding to this concern is the tendency of some surgeons to not take the time necessary to recalibrate the entire system when a reference structure or fiducial is dislocated as described above. When this occurs, the virtual image created by the imaging system is not a true reflection of the actual position, orientation and relationship of the body parts, non-body components and other reference points. Proceeding with surgical procedures with a coordinate system under these conditions can lead to obvious dangers.

### SUMMARY

Various aspects and embodiments of the present invention include frame attachments with portions that, when displaced or dislodged, will readily disconnect from a base secured to the reference point in the coordinate system and be able to be precisely repositioned.

According to one aspect of the present invention, a frame attachment includes a connecting portion with an interface designed to complement the receiving portion of a base secured in the coordinate system. The attachment device creates a stable connection with the base but, when displaced or dislodged, separates from the base without resulting in a change of location of the base within the coordinate system. The attachment can therefore be replaced without having to recalibrate the entire system.

According to another aspect, a frame attachment includes a connecting portion with an interface which is designed to complement a receiving portion of a base. The attachment device creates a stable connection with the base through the use of an additional connection aid, such as magnetic attraction, adhesive, hook and pile connectors, or any other material or force which creates a bond between the attachment device and base. The failure strength of the bond is preferably smaller than the failure strength of any portion of the attachment or the base. When the attachment device is displaced or dislodged, it separates from the base without resulting in a change of location of the base within the coordinate system. As such, the attachment device can be replaced without having to recalibrate the entire system.

According to other aspects of the present invention, the attachment device comprises fiducials, reference transmitters and/or other reference devices.

According to other aspects of the present invention, the base comprises a bone screw and/or other devices connected to a human body.

According to other aspects of the present invention, attachment devices and modular fiducials exhibit modularity such that they may be moved within a coordinate system without the disruption of the base secured within the coordinate system.

### BRIEF DESCRIPTION OF THE DRAWINGS

FIG. 1 shows a schematic side view of a modular fiducial according to one embodiment of the present invention.

FIG. 2 shows a schematic top view of the portion of a base having the fault interface for connection with the modular fiducial of FIG. 1.

FIG. 3 shows a perspective view of the modular fiducial of FIG. 1.

FIG. 4 shows a perspective view of the portion of the base having the fault interface of FIG. 2.

FIG. 5 shows a schematic view of the modular fiducial of FIG. 1 positioned for placement within the portion of the base having the fault interface of FIG. 2.

FIG. 6 shows a perspective view of an attachment device positioned for placement on top of a base according to another embodiment of the invention.

FIG. 7 shows a perspective view of an attachment device connected to a base according to another embodiment of the invention.

FIG. 8 shows a perspective view of an attachment device connected to a base according to still another embodiment of the present invention.

FIG. 9 shows a perspective view of a drill attachment according to another embodiment of the present invention positioned for connection to a bone screw.

FIG. 10 shows another perspective view of a drill attachment device of FIG. 9 positioned for placement in a bone screw.

FIG. 11 shows a perspective view of an attachment device according to another aspect of the present invention connected to a bone screw.

FIG. 12 shows a schematic view of a tracking system according to another embodiment of the present invention.

### DETAILED DESCRIPTION OF THE INVENTION

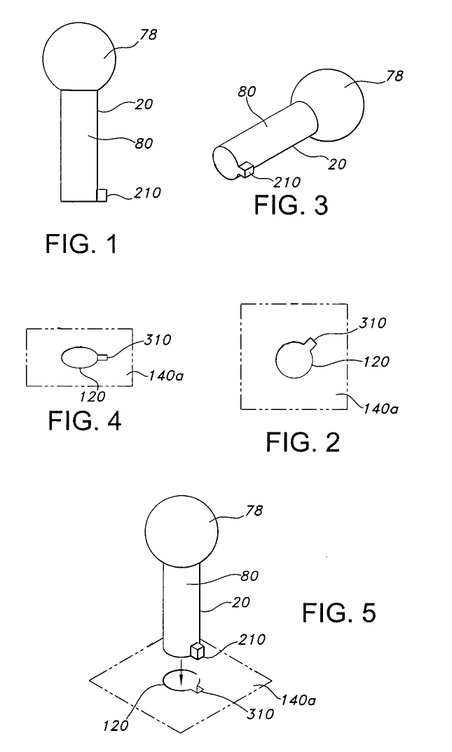

FIGS. 1-5 illustrate one form of device according to one embodiment of the present invention. FIGS. 1 and 3 show a modular indicium 20 that includes a fiducial or reflective element 78, a stem 80, and a key 210. The indicium 20 can instead be a transponder using any energy within the energy spectrum as desired, or any other active or passive device which is able to impart position information to another device so that, when that device senses position of three or more indicia 20 rigidly attached to a body part, tool, implant, trial or other thing in the operating room, the device is able to generate position and orientation information about the thing. The indicium can be of any desired shape, size, structure, material, circuitry such as RFID, or any other physical instantiation. The device which senses the indicium 20 can be any of the conventional or unconventional computer aided surgery systems mentioned above or otherwise, which include an imager for sensing the position and location of the indicium 20, computer functionality for generating position and orientation information about the thing to which the indicium is attached, and a display device which can render the thing correctly located and oriented according to position of the indicia 20.

In the embodiment shown in these figures, the key 210 protrudes from the lower portion of the stem 80. Any structure can be used to create a fault interface that has a failure strength less than the failure strength of the indicium to reference frame connection, or the reference frame to body part or other thing connection, or the failure strength of any part of these components or relevant parts of them. Preferably, the fault interface permits the indicium to be repositioned with respect to the thing or item in only one position and orientation if inadvertently or otherwise dislodged. That position is the position in which the indicium was originally registered into the computer aided surgery system. The present invention includes, however, any fault interface that permits the indicium to be repositioned without the need to reregister the indicium in the system.

FIGS. 2 and 4 show a base 140a with a fault interface 120 for the modular fiducial 20. The base may include, without limitation, a pin, a plate, a platform, or any other device which is secured within a reference system. The fault interface 120 has a groove 310 for placement of the key 210. This key/groove arrangement requires that the fiducial 20 be positioned in only one orientation in order to fit correctly. As a result, when the fiducial is dislodged or displaced relative to the base, either by purpose or accident, it may be replaced within the base in the precise location, position and orientation as its original placement in the coordinate system thus removing the necessity for the recalibration of the entire reference system. Placement of the fiducial 20 onto the base 140a is depicted in FIG. 5.

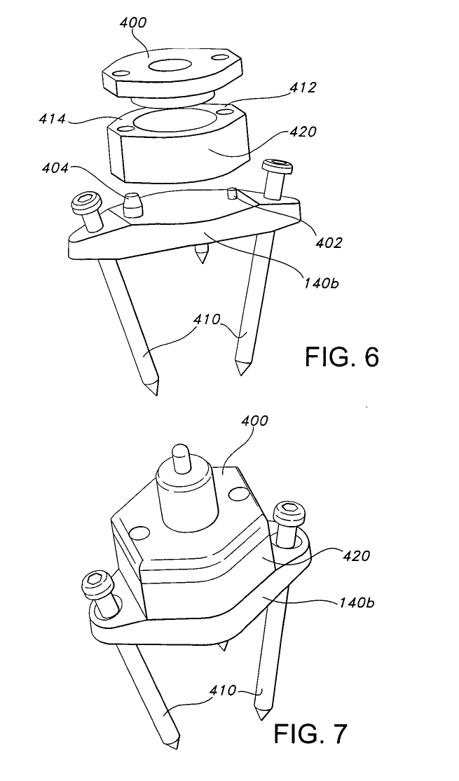

While FIGS. 1-5 depict one embodiment of the present invention, the invention includes any interface that allows registration of indicium or an attachment device with a base which allows the indicium or attachment device to be repositioned without the need to reregister the indicium in the system. For instance, FIGS. 6-8 depict other structures according to other embodiments of the present invention.

FIG. 6 shows an embodiment of the present invention in which the base 140b is in the form of a plate. The plate is securely attached to a body part or other reference point through the use of pins 410. In this embodiment, the base 140b includes two protrusions 402, 404 at the fault interface—a first protrusion 402 and a second protrusion 404. The protrusions are preferably of different size and/or shape, in order to allow another component to be attached in only one orientation. An attachment device 420 is included in this particular structure, which is designed to accept an additional element 400 for placement of a reference frame, fiducial or fiducials or other reference device or devices whether active or passive. The reference structure 420 includes two apertures 412, 414 which correspond in size and shape to protrusions 402, 404, whether or not those protrusions are of different size and/or shape. The design and placement of the protrusions and apertures preferably mandates that the attachment device 420 connects with the base 140b in only one position and orientation. Preferably, there is a friction fit at the fault interface which has a failure strength less than the failure strength of any part of, or relevant parts of any of components 140b, 400, or 420, and also less than the deformation limit or failure strength of the connection between the base 140b and the patient. Accordingly, when a fiducial, reference frame or other structure attached or connected, directly or indirectly to component 400 or 420 is dislodged or displaced, the attachment device 420 dislocates at the fault interface, but the base 140b remains securely in place. Because the design of the attachment device 420 and the base 140 allow connection in only position and orientation, however, the attachment device 420 may be precisely replaced on the base 140b and no further calibration is necessary before proceeding with surgery.

FIG. 7 shows a variation of the embodiment of FIG. 6 in which the attachment device 420 has been placed upon the base 140b. This embodiment includes an element 400 which can feature an active position indicating device or fiducial projecting above the surface of the element 400.

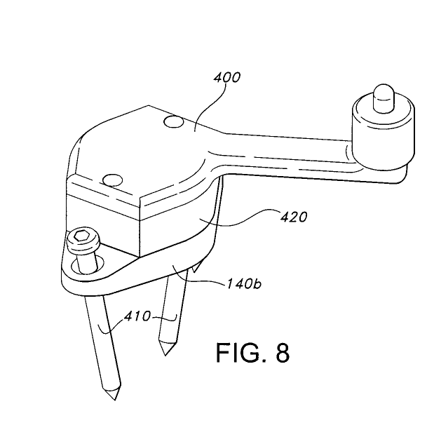

FIG. 8 shows yet another embodiment of the present invention. In this embodiment, the fiducial-accepting element 400 places the indicating device or fiducial outside the perimeter of the attachment device 420. However, the design of the attachment device 420 and the base 140b are such that, when sufficient force is exerted, the attachment device 420 dislodges while the base 140b remains securely in place allowing the attachment device 420 to be replaced in the same position and orientation. Therefore, the recalibration of the coordinate system is not necessary.

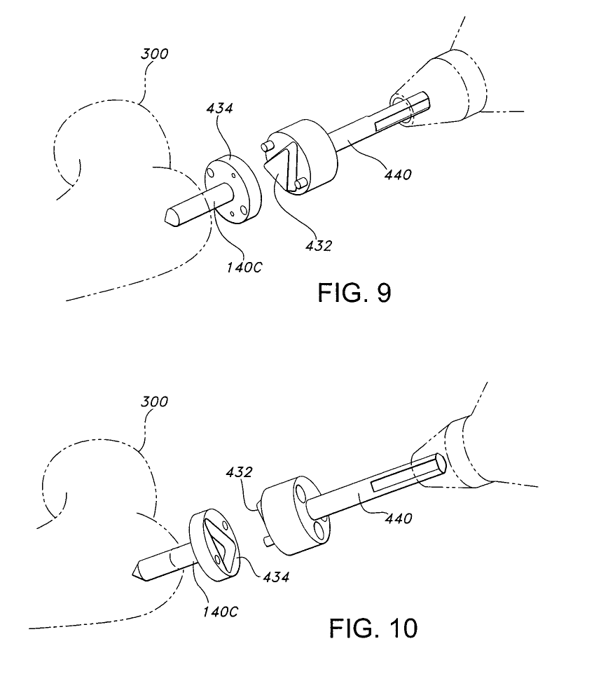

FIGS. 9 and 10 show another embodiment of the present invention in which the base 140c is in the form of a bone screw. The bone screw contains a fault interface 434 which corresponds to a pattern 432 on a drill attachment 440. This pattern is also present on the portion of the fiducial or other reference structure which attaches to the bone screw 140c. The interface on the bone screw 434 and corresponding pattern 432 require that the drill attachment 440 be positioned in only on orientation in order to fit correctly. The drill attachment 440 is connected to the bone screw 140c and the drill is used to secure the bone screw 140c to the bone 300.

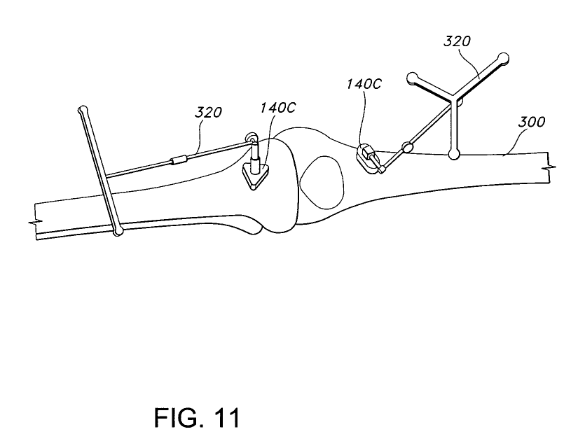

FIG. 11 shows a variation of the embodiment of FIGS. 9 and 10 in which attachment devices 320 have been placed on the bone screws 140c which are connected to a bone 300. The design of the attachment device 320 and the base 140c are such that, when sufficient force is exerted, the attachment device 320 dislodges while the bone screw 140c remains securely in place allowing the attachment device 320 to be replaced in the same position and orientation. Therefore, the recalibration of the coordinate system is not necessary.

According to certain embodiments of the present invention, a connection aid provides further support for the connection between the fiducial 20 and the base 140a,b,c. The connection aid may be located near the bottom portion of the fiducial 20, within the fault interface 120, both, or otherwise, and can include magnetic attraction, adhesives, hook and pile connectors, or any other materials or forces which result in a bond between the fiducial 20 and base 140a,b,c which features a smaller failure strength than relevant portions of either the fiducial or base. Accordingly, when sufficient force is placed on the fiducial 20, the connection aid allows the base to be displaced or dislodged in a manner that allows ready replacement into correct position and orientation.

In use, attachment devices 20, 320, or 420 bearing fiducials and/or active devices are connected to relevant body parts or part of tools, trials, implant components, tables, or other tangible things in the operating room. The fiducials and/or active devices are then registered into the computer aided surgery system in accordance with techniques discussed at length in the documents cited and incorporated by reference above. During surgery, the fiducials and/or active devices allow images of the thing to which they are attached to be represented in accurate position and orientation on a monitor with the aid of computer processing. However, when a fiducial or active device is inadvertently struck with an elbow or implement in a manner that would otherwise deform it in position or orientation or both, or dislodge it the thing to which was attached, instead the fault interface fails and allows the fidicial or active device or reference frame to be dislodged in a manner that permits its ready replacement in a manner that eliminates the necessity to reregister the indicium or the reference frame into the system. For example, the fiducial 20 may be replaced in its correct position, location and orientation with respect to the thing to which it was attached.

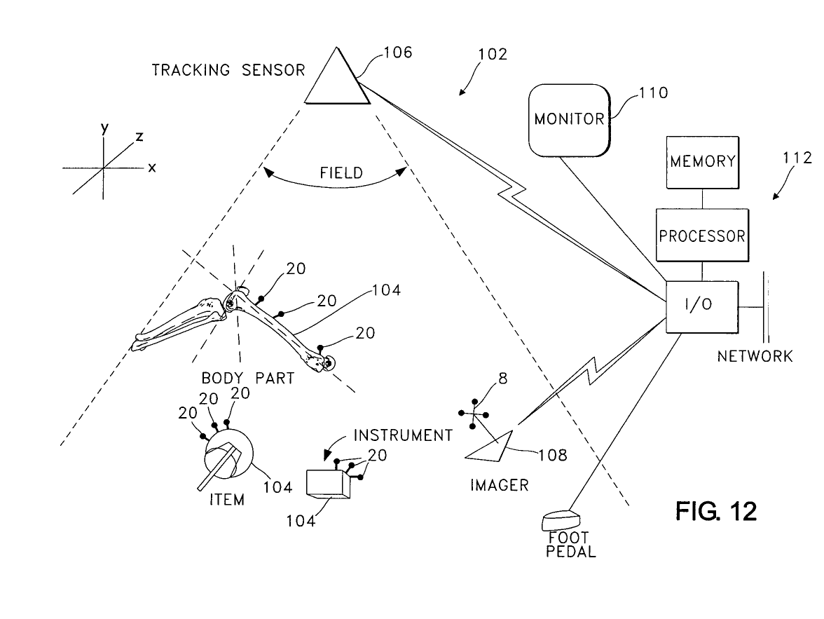

FIG. 12 shows a tracking system 102 that may utilize modular indicium 20 to track the orientation and/or position of desired items 104 within the tracking sensor's 106 field of vision. Modular indicium 20 or other reference structures 8 may be placed on items 104 to be tracked such that a tracking system 102 can track the position and/or orientation of any desired item in the field of view of the tracking sensor 106. The tracking sensor 106 may relay the position and/or orientation data to a processing functionality 112 which can correlate the data with data obtained from an imaging device 108 and output that data to a suitable output device 110.

The foregoing is provided for purposes of disclosure of various aspects and embodiments of the present invention. Changes, deletions, additions or and substitutions may be made to components, combinations, processes, and embodiments disclosed in this document without departing from the scope or spirit of the invention.

1. A computer aided surgery navigation system comprising:
a. a sensor adapted to sense position of a plurality of indicia attached by a reference frame to an item used in surgery;
b. computer functionality adapted to receive information from the sensor about position of the indicia and generate information corresponding to position and location of the item to which the indicia are attached;
c. rendering functionality adapted to render an image of the item correctly positioned and oriented in correspondence with the position of its indicia as sensed by the sensor;
d. wherein at least one of the indicia is attached to the item using a fault interface which features a failure strength smaller than the failure strength of the reference frame or connection between the reference frame and item, such that force exceeding the failure strength of the fault interface causes the fault interface to fail and the indicium to become dislodged relative to the item; and
e. wherein the fault interface features a structure which allows the indicium to be repositioned without the need to reregister the indicium in the system after the interface has failed.

2. A system according to claim 1 in which at least some of the indicia are fiducials.

3. A system according to claim 2 in which at least some of the fiducials feature reflective surfaces adapted to be sensed by an infrared sensor device.

4. A system according to claim 1 in which at least some of the indicia are active devices.

5. A system according to claim 4 in which at least some of the active devices are transponders which emit energy when interrogated.

6. A system according to claim 1 in which the fault interface is an asymmetrical structure.

7. A system according to claim 1 in which a plurality of indicia are connected to the item, using a single fault interface.

8. A system according to claim 1 in which a plurality of indicia are connected to the item, using a plurality of fault interfaces.

9. A system according to claim 1 in which a plurality of indicia are connected to the item, using a fault interface corresponding to each indicium.

10. A system according to claim 1 in which the fault interface includes structure adapted to create a friction fit.

11. A system according to claim 1 in which the item is a human body part.

12. A system according to claim 1 in which the item is a bone screw.

13. A system according to claim 1 in which the item is an implant.

14. A system according to claim 1 in which an additional connection aid is used to attach the indicia to the item.

15. A system according to claim 14 in which the additional connection aid is magnetic attraction.

16. A system according to claim 14, in which the additional connection aid is adhesive.

17. A system according to claim 14, in which the additional connection aid is hook and pile connectors.

18. A system according to claim 1 in which the indicium can be repositioned in only one position and orientation relative to the item after the interface has failed.

19. A computer aided surgery navigation system comprising:
a. an infrared sensor adapted to sense position of a plurality of fiducials attached by a reference frame to an item used in surgery;
b. computer functionality adapted to receive information from the sensor about position of the fiducials and generate information corresponding to position and location of the item to which the fiducials are attached;
c. rendering functionality adapted to render an image of the item correctly positioned and oriented in correspondence with the position of its fiducials as sensed by the sensor;
d. wherein at least one of the fiducials is attached to the item using a fault interface which features a failure strength smaller than the failure strength of the reference frame or connection between the reference frame and item, such that force exceeding the failure strength of the fault interface causes the fault interface to fail and the fiducial to become dislodged relative to the item; and
e. wherein the fault interface features a structure which allows the fiducial to be repositioned without the need to reregister the fiducial in the system after the interface has failed.

20. A system according to claim 19 in which the item is a body part.

21. A system according to claim 19 in which the item is a bone screw.

22. A system according to claim 19 in which the item is an implant.

23. A system according to claim 19 in which the fault interface is an asymmetrical structure.

24. A system according to claim 19 in which the fault interface includes a key and corresponding slot.

25. A system according to claim 19 in which the fault interface includes structure adapted to create a friction fit.

26. A system according to claim 19 in which an additional connection aid is used to attach the fiducial to the item.

27. A system according to claim 26 in which the additional connection aid is magnetic attraction.

28. A system according to claim 26 in which the additional connection aid is adhesive.

29. A system according to claim 26 in which the additional connection aid is hook and pile connectors.

30. A system according to claim 19 in which the fiducial can be repositioned in only one position and orientation relative to the item after the interface has failed.

31. A device for use in a computer aided surgical navigation system, the system comprising a sensor adapted to sense position of a plurality of indicia attached by a reference frame to an item used in surgery; computer functionality adapted to receive information from the sensor about position of the indicia and generate information corresponding to position and location of the item to which the indicia are attached; and rendering functionality adapted to render an image of the item correctly positioned and oriented in correspondence with the position of its indicia as sensed by the sensor; the device comprising a reference frame adapted to be connected to the item, at least one indicium connected to the reference frame, and a fault interface interposed between at least one indicium and the item; wherein the fault interface features a failure strength smaller than the failure strength of the reference frame or connection between the reference frame and item, such that force exceeding the failure strength of the fault interface causes the fault interface to fail and the indicium to become dislodged relative to the item; and wherein the fault interface features a structure which allows the indicium to be repositioned without the need to reregister the indicium in the system after the interface has failed.

32. A process for conducting computer aided surgery, comprising:
I. providing a computer aided surgery system, comprising:
a. a sensor adapted to sense position of a plurality of indicia attached by a reference frame to an item used in surgery;
b. computer functionality adapted to receive information from the sensor about position of the indicia and generate information corresponding to position and location of the item to which the indicia are attached;
c. rendering functionality adapted to render an image of the item correctly positioned and oriented in correspondence with the position of its indicia as sensed by the sensor;
d. wherein at least one of the indicia is attached to the item using a fault interface which features a failure strength smaller than the failure strength of the reference frame or connection between the reference frame and item, such that force exceeding the failure strength of the fault interface causes the fault interface to fail and the indicium to become dislodged relative to the item; and
e. wherein the fault interface features a structure which allows the indicium to be repositioned without the need to reregister the indicium in the system after the interface has failed;
II. registering the indicia into the system;
III. navigating the item during surgery using the image rendered by the rendering functionality;
IV. dislodging at least one indicium in a manner that causes a fault interface to fail;
V. repositioning the indicium into correct position and orientation relative to the item;
VI. continuing to navigate the item during surgery without the need to reregister the indicium into the system.
